(19) United States
(12) Patent Application Publication
(10) Pub. No.: US 2016/0151161 A1
(43) Pub. Date: Jun. 2, 2016

(54) BONE REPLACEMENT MATERIAL AND METHOD FOR PRODUCING BONE REPLACEMENT MATERIAL

(71) Applicants: CERAMTEC GMBH, Plochingen (DE); FRIEDRICH ALEXANDER-UNIVERSTTÄT ERLANGEN- NÜRNBERG, Erlangen (DE)

(72) Inventors: Kathrin Lorenz, Buchs (CH); Tobias Fey, Erlangen (DE); Peter Greil, Weisendorf (DE); Heinrich Wecker, Eckental (DE); Alfons Kelnberger, Röthenbach (DE)

(21) Appl. No.: 14/900,430

(22) PCT Filed: Jun. 25, 2014
(86) PCT No.: PCT/EP2014/063428

§ 371 Date: Dec. 21, 2015

(30) Foreign Application Priority Data
Jun. 27, 2013 (DE) 102013212455.4

(51) Int. Cl. A61F 2/30; B28B 1/26; B28B 1/24; A61F 2/44; B28B 1/00

(52) U.S. Cl. CPC A61F 2/30771; A61F 2/4455; A61F 2/4425; B28B 1/001; B28B 1/24; B28B 1/26; A61F 2002/3092; A61F 2002/3093; B33Y 10/00

(57) ABSTRACT

A ceramic bone replacement material and to a generative method for producing said bone replacement material, in particular ceramic bone replacement materials used in implants and preferably in spinal column implants. Generative manufacturing methods make it possible to shape the structures of the bone replacement.

The invention relates to a ceramic bone replacement material and to a generative method for producing said bone replacement material. In particular, the invention relates to ceramic bone replacement materials used in implants and preferably in spinal column implants. Generative manufacturing methods make it possible to shape the structures of the bone replacement material.

Endoprosthetic components, for example for fusing vertebrae (cages), are known. The shape thereof is adapted to the anatomy of the human vertebra, they are placed between two vertebrae and replace the intervertebral disc either completely or in part. In addition to replacing the intervertebral discs, it is also possible to replace entire spinal column segments, for example vertebrae and adjacent intervertebral discs, with cages.

In a first phase of their time in the human body, the spinal column implants typically keep adjacent vertebrae spaced apart, in an anatomically correct and neurologically optimum position, by means of their mechanical properties alone. In a second phase, they encourage the two surrounding vertebrae to fuse and therefore to grow together.

Known ceramic cages are generally annular in shape or are adapted to the shape and anatomy of the human vertebrae, the ring being made of a monolithic, i.e. dense, highly resilient and rigid ceramic.

Said cages have a cavity in the centre which can be filled with known bone replacement materials (autologous or allogenic). It is further possible to fill said cavity with a synthetic porous osteoinductive or osteoconductive core structure. In ceramic cages, the synthetic core structure can be based on the same type of ceramic materials, different ceramic materials or non-ceramic materials and is generally significantly less rigid than the outer ring. In this region, bone cells are intended to form new bone material, the participating cells requiring a corresponding mechanical stimulus.

For these structures, the term “bone replacement material” is used synonymously with the term “porous core” or “porous core structure,” which is expedient in particular with regard to the spinal column implants described in this case. The same applies to the expression “casing” or “casing structure” which are used to denote individual embodiments of the monolithic load-bearing material. However, the use of said expressions is not intended to restrict the invention to spinal column implants. The spinal column implants should merely be seen as preferred embodiments or preferred fields of application for the bone replacement material according to the invention. In principle, the bone replacement material according to the invention can be used wherever bones are intended to grow either together with or into an implant.

According to a preferred embodiment of the invention, a porous ceramic bone replacement material comprises an open continuous porosity of at least 25 vol. %.

Different methods are known for producing said porous core structures.

The synthetic core structure can be produced by means of a foaming method, in which a gas is introduced into a ceramic slip in order to produce bubbles. Said structures are mechanically relatively stable and resilient; the compressive strengths are in the double-digit megapascal range.

However, it is disadvantageous that the pores produced by gas foaming are usually closed. The porous structures are either not interconnecting or are barely interconnecting and therefore essential requirements for the formation of new bone, specifically the permeability to fluids and ingrowth paths for bones cells, are not met.

Another variant of the targeted pore formation in ceramic structures is based on the use of pore generators, for example organic beads which are introduced into or applied to the ceramic body in a targeted manner during the process. The porn generators are then burnt off and leave pores.

This technology is suitable for forming rough surfaces which in fact provide effective conditions for bones to fuse and grow. The porosity produced by this method is, however, substantially non-interconnecting, i.e. it is not suitable for ossifying a specific volume with a bone substance. This technology therefore cannot be implemented in the use according to the invention in implants for fusing vertebrae.

Furthermore, the reticulated method (or “Schwarzwalder method”) for producing porous ceramics is known and is a specific moulding method. In this case, organic template bodies having a suitable open-pore structure, for example organic foam structures, are coated with ceramic slips and then subjected to the known ceramic thermal processes in order to ultimately produce an open-cell trabecular ceramic structure.

However, this method always produces hollow webs in the finished ceramic product since the organic template body is burnt off and hollow webs are left in place thereof. Although this method produces a porous structure, said structure only has low strengths in the single-digit megapascal range.

An additional disadvantage is that the structure of the porous ceramic is predetermined by the structure of the template body which does not necessarily correspond to the ideal biological conditions in the human body. The structure to be achieved can only be shaped in a manner restricted by the selection of a corresponding template body.

The object of the invention is therefore to prevent the disadvantages of the abovementioned methods and to provide a stable porous bone replacement material which can be used in particular in spinal column implants. Furthermore, methods for producing said bone replacement material are intended to be provided.

In particular, high-strength and damage-tolerant ceramic materials are intended to be used. The bone replacement materials are intended to provide the best possible conditions for the intergrowth of the implant with bone cells.

Oxide ceramic materials based on Al₂O₃, ZrO₂ or mixed ceramics consisting thereof, such as ZTA (zirconia toughened alumina), ATZ (alumina toughened zirconia) or ceramic composite materials comprising dispersoid phases are particularly suitable. Si₃N₄-based or SiC-based materials are also conceivable.

Generative manufacturing methods are used for manufacturing the spinal column implants. In this case, the following approaches can be followed in principle:
directly manufacturing the porous ceramic bone replacement material or a complete implant comprising a porous ceramic bone replacement material and a monolithic load-bearing material, by means of a generative method,
manufacturing negative moulds for producing the porous ceramic core structures (bone replacement material) or the entire integral structure of the implants, i.e. negative moulds for the outer ring and for the porous core structure, and subsequently manufacturing the ceramic implants using suitable ceramic shaping methods.

Irrespective of the principle approach, open-cell trabecular structures which are highly flexible with regard to possible shapes can be produced by means of generative manufacturing methods, which structures fulfil the biological requirements placed on bone cell growth in an optimum manner. The biological requirements relate to the production of bone-forming cells (osteoblasts) and vessel-forming cells (endothelium), for example.

The core structures according to the invention are highly advantageous due to the very flexible and complex possibilities in terms of the shape thereof. The fusion structure can have a graded porosity in terms of the number and/or size of the pores, for example. It is possible to shape pore moulds, such as directed channel structures which encourage the newly formed bone material to vascularise, in a targeted manner.

This not only ensures that bone material forms and grows but also that the vitality of the newly formed bone is provided and maintained.

There is significantly greater design freedom by comparison with conventional techniques, and therefore structures which are ideal for the bone cells can be provided.

For example, the natural and individual bone structure of a patient can be used as a template, on the basis of computer tomography data, in order to promote bone formation suited to the patient.

By comparison with the direct moulding methods, is it possible not only to minimise hollow webs, but also to completely avoid them. Dense webs can be produced so that a web having the same cross section has a significantly increased mechanical strength.

Another advantage is the possibility of reducing the cross section of the webs while keeping the same strength of the porous structures. A larger proportion of endogenous bone cells can thus be achieved in the core structure of the cage.

However, hollow webs can also be produced in a targeted manner, which webs have a round cross section and are therefore substantially more suitable for ceramic than triangular hollow webs which form when templates are burnt off. Edges having acute angles can be avoided. The targeted production of hollow webs can be advantageous, e.g. for filling the hollow webs with growth-promoting or antimicrobial substances.

It is possible to form horizontally or vertically extending supply channels, the size and diameter of which are adapted to the human capillary system.

When using CAD-based stereolithographic methods to form the porous structures, very high resolutions can be achieved irrespective of whether plastics-based negative moulds or ceramic positive structures are produced. Currently, resolutions of up to 30 μm in the z direction and up to 20 μm in the lateral x-y direction are realistic.

A further advantage is the high reproducibility when forming the porous structures.

In addition, the structures can be produced such that, once finished, they can be optimally cleaned and sterilised, which is extremely important in a medical product.

If negative moulds, for example made of photo-curable plastics materials (photopolymers), are produced, said moulds can be infiltrated with a ceramic body, for example a slip or an injection moulding compound, and then further processed to form ceramic cages, either in a modular or integrated manner. Suitable methods are, inter alia, slip casting and injection moulding, in particular low-pressure injection moulding (hot-moulding or LIM).

An implant comprising a porous ceramic bone replacement material and a monolithic load-bearing material can be readily produced by means of a generative method of this type. The monolithic load-bearing material is also preferably made of ceramic, in particular preferably made of the same type of ceramic as the porous bone replacement material.

The bone replacement material and the monolithic load-bearing material can both preferably be moulded by means of ceramic injection moulding.

If an LIM method is used, the process sequence can for example be as follows: A generatively produced polymer core is placed in a mould which determines the shape of the spinal column implant and the ceramic low-pressure injection moulding compound is injection-moulded there around at temperatures of over 100° C.

According to the known prior art, this compound can be produced from the ceramic powder and processed together with waxy organic matter.

A template core (negative mould) for example produced from supporting polymers by means of photolithography or fused deposition modelling (FDM), determines the pore structure. The template core is removed, after impregnation with the ceramic, for example by means of melting out, dissolving or thermal decomposition.

When subsequently debinding and sintering in air, the carbon-containing core which may remain can be removed by means of oxidation at over 600° C. A specific ceramic trabecular structure can therefore be produced either together with or separately from a monolithic casing region for a spinal column implant.

In addition to the flexible shaping of the geometry of the core structure in said cages, the significant advantage here is that the cage is shaped in one work step.

In generative methods, the a) direct methods (3D powder bed printing, ceramic inkjet direct printing) and the b) indirect methods (FDM, stereolithography, in particular CAD-based stereolithography) are preferably used.

The resolution limits of the respective methods have been converted into pore sizes and web widths according to the current level of knowledge, and are shown in the table:

| Method | Pore size | | Web widths | |
|---|---|---|---|---|
| 3D powder bed printing | [symbol] 300 | μm | [symbol] 300 | μm |
| Ceramic inkjet printing | [symbol] 15 | μm | [symbol] 15 | μm |
| FDM | [symbol] 250 | μm | [symbol] 250 | μm |
| Stereolithography | [symbol] 50 | μm | [symbol] 50 | μm |

10 mm can be taken as the maximum commercial upper limit for the pore sizes and web widths. Large pore sizes are advantageous, for example, when channel structures are intended to be laid.

An example of a suitable generative or rapid prototyping method having a highly flexible geometric design is the FDM method (fused deposition modelling), in which models of negative moulds are made from thermoplastic polymeric wire which is led to a nozzle where it is heated to just below its melting point. The semi-liquid thermoplastic material is then applied to a pre-existing layer as an additional layer where it is immediately cooled again. The layers adhere to one another because the liquid plastics material fuses to the pre-existing layer. Possible materials are ABS (acrylonitrile butadiene styrene), PLA (polyacetide) or PVA. The polymer structure is removed thermally by means of decomposition when using ABS and PLA. The water-soluble PVA can be dissolved by treatment in a water bath at temperatures of lower than 60° C.

Irrespective of the production method selected, the core structures produced can be connected, either integrally by being combined with the casing structure in the green state and being subsequently sintered, or in an interlocking manner by being combined, in the sintered state, with the casing structure, which is independently manufactured.

Epoxy materials having a softening point above the softening point of the LIM injection moulding compound, preferably above 120° C., have proven to be suitable materials for semi-permanent cores or negative moulds.

The negative moulds can also be infiltrated with ceramic bodies which contain pore generators. Once the semi-permanent core and the pore generators have been removed, said ceramic bodies result in an additional microporosity. By setting a bimodal pore size distribution in this way, the two aspects of
optimum structure for bone cells, and
vascularisation

can be allowed for in the best possible manner.

A second variant involves the aforementioned stereolithographic methods, in which photo-curable polymers are used which are hardened and stabilised layer by layer under exposure to UV light.

As a third variant, direct production by means of commercial 3D printing of ceramic powders (manufacturer e.g. Z-Corp) can be used to produce the trabecular structures. In this case, the 3D component is produced from ceramic powders, optimised for the printing process (particle size and distribution, and proportion of binder), by means of a layered formation in an inkjet printing process using a liquid binder. Powder regions which are not printed with the liquid can be removed by blowing out or by manual processing once the component has hardened in the construction chamber. The component has to be dimensioned such that it is possible to remove the residual powder. In particular, an open porosity is essential. The printable layer thickness in the z direction depends on the particle size distribution of the powder and the resolution of the device. The layer thickness is typically between 125 and 150 μm. The xy resolution corresponds to the resolution of the layer thickness since it is also dependent on the particle size distribution.

Ceramic direct printing by means of standard inkjet technology can be used as the fourth variant. In this case, an optimised ceramic slip (average particle size <100 nm) is printed directly onto a substrate in layers.

This is advantageous in that the direct formation of ceramic structures is possible. In this case, the ceramic starting powders are replaced with photo-curable polymers, making the formation or stereolithographic shaping possible. The photo-cured polymers are then burnt off.

A significant advantage of the first approach (for example FDM) is that a cage having a trabecular structure can thereby be produced in one work step and no additional outlay is required to integrally bond or interlockingly connect it to the dense casing structure of the cage.

All of these methods are suitable not only for cages but also for producing partial joint endoprostheses (e.g. partial resurfacing) or generally for producing bone replacement material.

1.-13. (canceled)

14. A method for producing a porous ceramic bone replacement material comprising producing the porous ceramic bone replacement material with a generative manufacturing method.

15. A method according to claim 14, wherein the generative method is selected from the group consisting of 3D powder bed printing, ceramic inkjet printing, fused deposition modelling and CAD-based stereolithography.

16. A method according to claim 14, wherein the generative method is used to produce a negative mold of the porous bone replacement material.

17. A method according to claim 16, further comprising the steps of
infiltrating the negative mold is infiltrated with a ceramic body, and
removing the negative mold is removed and the ceramic body is sintered.

18. A method according to claim 17, wherein the negative mold is removed by melting out, dissolving or thermal decomposition.

19. A method according to claim 17, wherein the ceramic body contains pore generators.

20. A method according to claim 14, wherein the bone replacement material is directly produced by the generative method.

21. A method for producing an implant comprising a porous ceramic bone replacement material which is produced according to claim 14, wherein the porous bone replacement material is combined with a monolithic load-bearing material to form an implant.

22. A method according to claim 21, wherein the monolithic load-bearing material is made of ceramic.

23. A method according to claim 22, wherein the bone replacement material and the monolithic load-bearing material are both molded in one work process by ceramic injection molding.

24. A method according to claim 22, wherein the ceramic bone replacement material and the monolithic load-bearing material are integrally bonded by joint sintering.

25. A method according to claim 22, wherein the bone replacement material and the monolithic load-bearing material are produced independently of one another and are then connected in an interlocking manner.

26. An implant produced by producing a porous ceramic bone replacement material comprising producing the porous ceramic bone replacement material with a generative manufacturing method, and forming an implant from the porous ceramic bone material, wherein the implant is a spinal column implant, cage or partial joint endoprosthesis.

27. A method according to claim 23, wherein the ceramic bone replacement material and the monolithic load-bearing material are integrally bonded by joint sintering.
